(19) United States
(12) Patent Application Publication
(10) Pub. No.: US 2006/0122603 A1
(43) Pub. Date: Jun. 8, 2006

(54) HYBRID BONE SCREW AND PLATE SYSTEMS

(75) Inventor: Eric Kolb, Milton, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DEPUY SPINE, INC., Raynham, MA (US)

(21) Appl. No.: 10/904,990

(22) Filed: Dec. 8, 2004

(51) Int. Cl. A61F 2/30

(52) U.S. Cl. 606/69

(57) ABSTRACT

Various exemplary spinal plating systems are provided having a spinal plate with at least one thru-bore formed therein for interchangeably receiving at least two different bone screws, thus allowing a surgeon to select a desired construct depending upon the intended use. In one exemplary embodiment, the spinal plating system includes a fixed angle bone screw and a variable angle bone screw.

### BACKGROUND

For a number of known reasons, bone fixation devices are useful for promoting proper healing of injured or damaged vertebral bone segments caused by trauma, tumor growth, or degenerative disc disease. The fixation devices immobilize the injured bone segments to ensure the proper growth of new osseous tissue between the damaged segments. These types of bone fixation devices often include internal bracing and instrumentation to stabilize the spinal column to facilitate the efficient healing of the damaged area without deformity or instability, while minimizing any immobilization and post-operative care of the patient.

One such device is an osteosynthesis plate, more commonly referred to as a bone fixation plate, that can be used to immobilize adjacent skeletal parts such as bones. Typically, the fixation plate is a rigid metal or polymeric plate positioned to span bones or bone segments that require immobilization with respect to one another. The plate is fastened to the respective bones, usually with bone screws, so that the plate remains in contact with the bones and fixes them in a desired position. Bone plates can be useful in providing the mechanical support necessary to keep vertebral bodies in proper position and bridge a weakened or diseased area such as when a disc, vertebral body or fragment has been removed.

Such plates have been used to immobilize a variety of bones, including vertebral bodies of the spine. These bone plate systems usually include a rigid bone plate having a plurality of screw openings. The bone plate is placed against the vertebral bodies and bone screws are used to secure the bone plate to the spine, usually with the bone screws being driven into the vertebral bodies.

Bone screws can be supported in a spinal plate in either a fixed angle or a variable angle fashion. In a fixed angle fashion, the bone screws are not permitted to move angularly relative to the plate. Conversely, in a variable angle fashion, the bone screws can move relative to the plate. The use of fixed angle and variable angle bone screws allows the surgeon to select the appropriate bone screw based on the particular treatment. While current plating systems can be effective, they typically require the use of different plates to obtain the desired bone screw fixation.

Accordingly, there remains a need for an improved plating system that allows the surgeon to use a single plate and to select between various types of bone screw fixation.

### SUMMARY

Disclosed herein are various exemplary spinal plating systems for use in treating spinal pathologies. The plating systems can be configured to receive at least two types of bone screws, such as a variable angle bone screw and a fixed angle bone screw, thus allowing a surgeon to select a desired construct depending on the intended use. While various techniques can be used to provide such a plating system, in one exemplary embodiment the plating system can include a spinal fixation plate having a thru-bore formed therein with proximal and distal regions that are adapted to selectively and interchangeably receive a variable angle bone screw and a fixed angle bone screw.

While the thru-bore in the spinal plate can have a variety of configurations, in one exemplary embodiment the distal region of the thru-bore can have a shape that complements the shape of a distal region of the head of each bone screw, and the proximal region of the thru-bore can have a shape that is adapted to engage a proximal region of a head of the fixed angle bone screw to substantially prevent movement of a shank of the fixed angle bone screw relative to the plate, and that is adapted to allow movement of a head of the variable angle bone screw to allow polyaxial movement of a shank of the variable angle bone screw relative to the plate. In one exemplary embodiment, the distal region of the thru-bore can be substantially spherical and the proximal region of the thru-bore can have a non-spherical shape, such as a substantially cylindrical shape, a substantially conical shape, or some other shape. In another exemplary embodiment, the proximal region of the thru-bore can have a flange-receiving recess formed therein for preventing movement of the fixed angle bone screw.

A variety of exemplary bone screws are also provided and the bone screws can be adapted to interchangably mate with a thru-bore in a spinal fixation plate. In one exemplary embodiment, a first bone screw, e.g., a variable angle bone screw, and a second bone screw, e.g., a fixed angle bone screw, are provided and the bone screws are adapted to be interchangeably received within the same thru-bore in a spinal fixation plate. For example, the variable angle bone screw can have a head that is receivable within a thru-bore in a spinal plate such that a shank of the screw is angularly variable relative to the plate, and the fixed angle bone screw can have a head with a shape that is complementary to a shape of the thru-bore in the plate to engage the thru-bore such that the shank is angularly fixed relative to the plate. While the head of each bone screw can have a variety of configurations, in one exemplary embodiment the head of the variable angle bone screw can have a substantially spherical shape, and the head of the fixed angle bone screw can have a substantially spherical distal region and a non-spherical proximal region. By way of non-limiting example, the non-spherical proximal region can be substantially cylindrical or substantially conical. In other embodiments, the proximal region can include a flange formed thereon that is adapted to be received within a flange-receiving recess in a thru-bore in a plate.

The various exemplary spinal fixation plates, variable angle bone screws, and/or fixed angle bone screws disclosed herein can also be provided as part of a spinal fixation kit.

### BRIEF DESCRIPTION OF THE DRAWINGS

FIG. 1A is a side perspective view of one exemplary embodiment of a spinal fixation plate having a bone screw coupled thereto;

FIG. 1B is a cross-sectional view of a thru-bore in the spinal fixation plate shown in FIG. 1A taken across line B-B;

FIG. 1C is a side view of one exemplary embodiment of a fixed angle bone screw that is adapted to mate with the thru-bore shown in FIG. 1B;

FIG. 1D is a side view of one exemplary embodiment of a variable angle bone screw that is adapted to mate with the thru-bore shown in FIG. 1B;

FIG. 1E is a cross-sectional view of the fixed angle bone screw of FIG. 1C and the variable angle bone screw of FIG. 1D disposed within two thru-bores of the spinal fixation plate shown in FIG. 1A;

FIG. 2A is a side view of another exemplary embodiment of a thru-bore of a spinal fixation plate;

FIG. 2B is a side view of another exemplary embodiment of a fixed angle bone screw that is adapted to mate with the thru-bore shown in FIG. 2A;

FIG. 2C is a cross-sectional view of the fixed angle bone screw of FIG. 2B and the variable angle bone screw of FIG. 1D disposed within a plate having two thru-bores configured as shown in FIG. 2A;

FIG. 3A is a side view of another exemplary embodiment of a thru-bore of a spinal fixation plate;

FIG. 3B is a side view of another exemplary embodiment of a fixed angle bone screw that is adapted to mate with the thru-bore shown in FIG. 3A; and

FIG. 3C is a cross-sectional view of the fixed angle bone screw of FIG. 3B and the variable angle bone screw of FIG. 1D disposed within a plate having two thru-bores configured as shown in FIG. 3A.

### DETAILED DESCRIPTION OF THE EXEMPLARY EMBODIMENTS

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Disclosed herein are various exemplary spinal plating systems having a spinal plate with at least one thru-bore formed therein for interchangeably receiving at least two different bone screws, thus allowing a surgeon to select a desired construct depending upon the intended use. In one exemplary embodiment, the spinal plating system can include a fixed angle bone screw and a variable angle bone screw, and at least one of the thru-bores in the plate can be adapted to receive a head of the variable angle bone screw such that a shank of the screw is movable, e.g., polyaxial, relative to the plate and to receive and engage a head of the fixed angle bone screw such that a shank of the screw is maintained in a substantially fixed position relative to the plate. A person skilled in the art will appreciate that the exemplary techniques used to achieve a system having two interchangeable bone screws can be incorporated into a variety of devices other than spinal plates, and that other bone engaging devices can be used instead of bone screws. Moreover, the exemplary spinal plating systems can include a variety of other features known in the art.

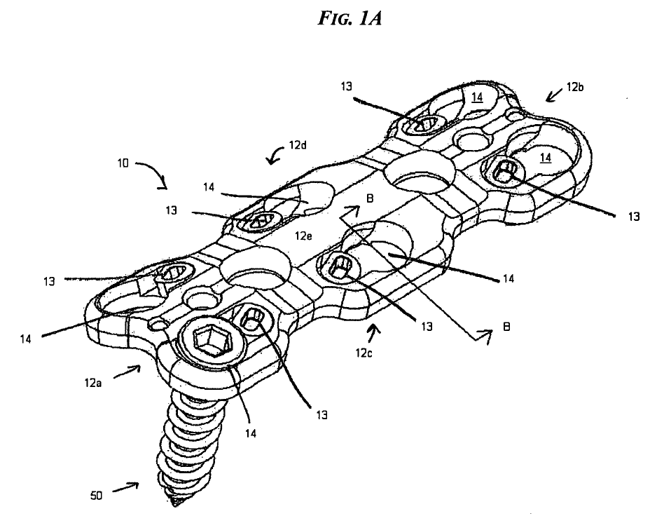

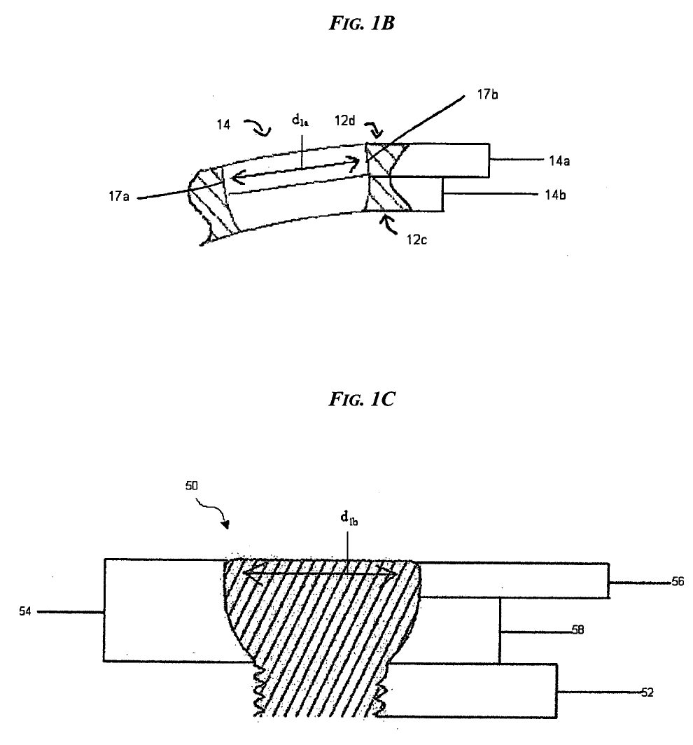

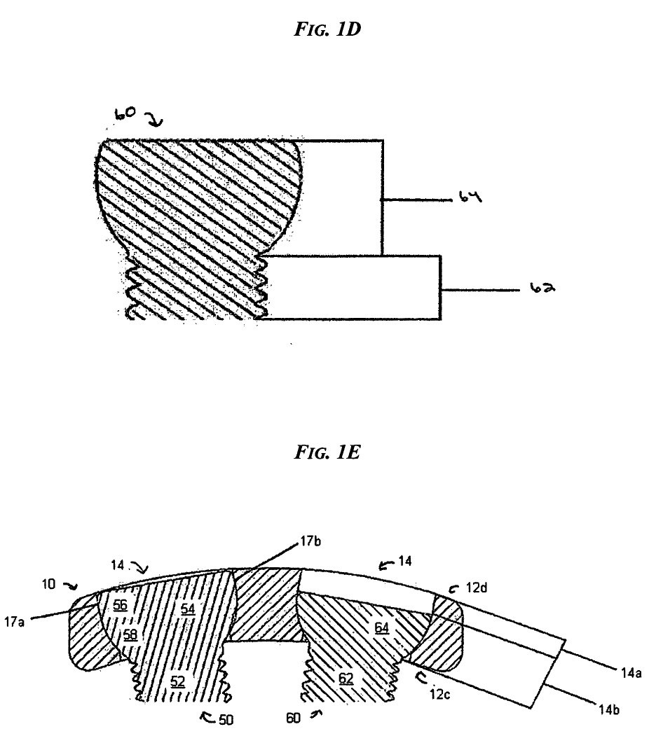

FIGS. 1A-1E illustrate one exemplary plating system having a spinal plate 10, a fixed angle bone screw 50, and a variable angle bone screw 60. The spinal plate 10 can have virtually any configuration, but in the illustrated exemplary embodiment it has an elongate shape with six thru-bores 14 formed therein and extending between opposed bone-contacting and non-bone-contacting surfaces 12e, 12f. The plate 10 can, however, include any number of thru-bores 14, and some or all of the thru-bores 14 can be the same or different from one another. For reference purposes, the plate 10 will be referred to as having opposed proximal and distal ends 12a, 12b connected by opposed first and second sides 12c, 12d. Each bone screw 50, 60 can also have a variety of configurations, but in the illustrated embodiment each bone screw 50, 60 includes a head 54, 64 that is adapted to be received within and interfaced with a thru-bore 14 in the plate 10, and a threaded shank 52, 62 that is adapted to engage bone to mate the plate 10 to bone.

Each thru-bore 14 in the plate 10 can have a variety of configurations, but in an exemplary embodiment at least one of the thru-bores 14 can be adapted to selectively and interchangeably receive the fixed angle bone screw 50 and the variable angle bone screw 60. In particular, at least one of the thru-bores 14 can be adapted to receive the head 64 of bone screw 60 in a variable angle construct such that the head 64 can pivot within the thru-bore 14 to allow the shank 62 of the screw 60 to move in multiple directions, e.g., proximal, distal, medial, lateral, and combinations thereof, such that the shank 62 is polyaxial relative to the plate 10. The thru-bore 14 can also be adapted to receive the head 54 of bone screw 50 in a fixed angle construct such that the head 54 is engaged by the thru-bore 14 to maintain the shank 52 of the screw 50 in a substantially fixed position relative to the plate 10. A person skilled in the art will appreciate that while the head 54 and the shank 52 are in a substantially fixed position, the bone screw 50 may toggle or have some micro-motion due to manufacturing tolerances.

While various techniques can be used to allow polyaxial movement of the variable angle bone screw 60 and to prevent movement of the fixed angle bone screw 50, FIG. 1B illustrates one exemplary embodiment of one of the thru-bores 14 in the plate 10. As shown, the exemplary thru-bore 14 can have a distal region 14b and a proximal region 14a for receiving a portion of the head 54, 64 of each bone screw 50, 60. The distal region 14b can have a shape that complements a shape of a corresponding portion of the head 54, 64 of each bone screw 50, 60, and the proximal region 14a can have a shape that complements a shape of a corresponding portion of the head 54 of the fixed angle bone screw 50, but that is different than a shape of a corresponding portion of the variable angle bone screw 60, or that otherwise does not interfere with movement of the variable angle bone screw 60, as will be discussed in more detail below. While the proximal region 14a of the thru-bore 14 can have a variety of shapes, in one exemplary embodiment the proximal region 14a can have a substantially cylindrical shape. In particular, the opposed walls 17a, 17b of the thru-bore 14 can be substantially parallel to one another such that the proximal region 14a of the thru-bore 14 has a constant diameter d₁ₐ. The distal region 14b of the thru-bore 14 can also have a variety of shapes, but in one exemplary embodiment the distal region 14b of the thru-bore 14 can have a substantially spherical shape as shown.

Each bone screw 50, 60 can also have a variety of configurations, but in one exemplary embodiment, shown in FIGS. 1C and 1D, the head 54 of the fixed angle bone screw 50 can have a shape that complements a shape of the proximal and distal regions 14a, 14b of the thru-bore 14, and the head 64 of the variable angle bone screw 60 can have a shape that complements the shape of the distal region 14b of the thru-bore 14, but that does not complement or otherwise engage the proximal region 14a of the thru-bore 14. Referring first to FIG. 1C, the head 54 of the fixed angle bone screw 50 can have a proximal region 56 with a substantially cylindrical shape and a distal region 58 with a substantially spherical shape, or some other shape that approximates a sphere. The substantially cylindrical proximal region 56 of the head 54 can vary in size, but in an exemplary embodiment the proximal region 56 has a size that is adapted to engage the proximal region 14a of the thru-bore 14. In particular, the proximal region 56 of the head 54 of the bone screw 50 can have a constant diameter d_{1b} that is only slightly less than the diameter d₁ₐ of the proximal region 14a of the thru-bore 14. As a result, when the bone screw 50 is disposed within the thru-bore 14, as shown in FIG. 1E, the proximal region 56 of the head 54 of the bone screw 50 will occupy and engage the proximal region 14a of the thru-bore 14 such that the shank 52 can be held in a substantially fixed position.

Conversely, in the embodiment shown in FIG. 1D, the head 64 of the variable angle bone screw 60 can have a configuration that does not engage the proximal region 14a of the thru-bore 14. In an exemplary embodiment, the head 64 can have a substantially spherical shape, or some other shape that generally approximates a sphere, such that the head 64 can be received within at least the distal region 14b of the thru-bore 14. The head 64 does not necessarily need to include a proximal region that is received within the proximal region 14a of the thru-bore 14, but to the extent that it does, the proximal region (not shown) of the head 64 can have a diameter that is smaller than the diameter d₁ₐ of the proximal region 14a of the thru-bore 14 to allow the head 64 to pivot relative to the thru-bore 14. By way of non-limiting example, the head 64 can include a substantially spherical proximal region such that a gap exists between the head 64 and the proximal region 14a of the thru-bore 14, thereby allowing polyaxial movement of the bone screw 60. FIG. 1E illustrates the substantially spherical head 64 of the bone screw 60 disposed within the substantially spherical distal region 14b of the thru-bore 14. The substantially spherical shape of the head 64 and the corresponding substantially spherical shape of the distal region 14b of the thru-bore 14 will allow the head 64 to pivot therein, thereby allowing polyaxial movement of the shank 62 relative to the plate 10.

A person skilled in the art will appreciate that the head 64 of the variable angle bone screw 60 can have a variety of shapes other than spherical. For example, the head 64 of the variable angle bone screw 60 can have a stepped configuration, an elliptical shape, a shape that approximates a sphere, or any other shape that allows the head 64 of the variable angle bone screw 60 to be angularly variable relative to the thru-bore 14.

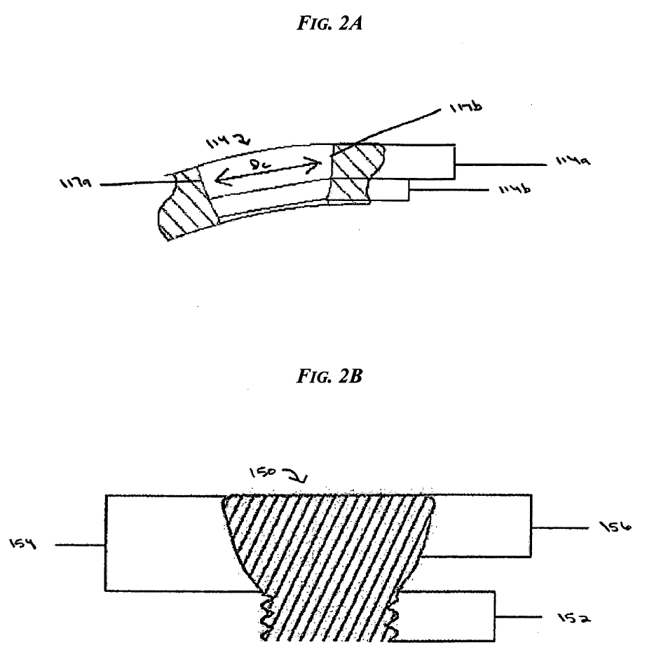

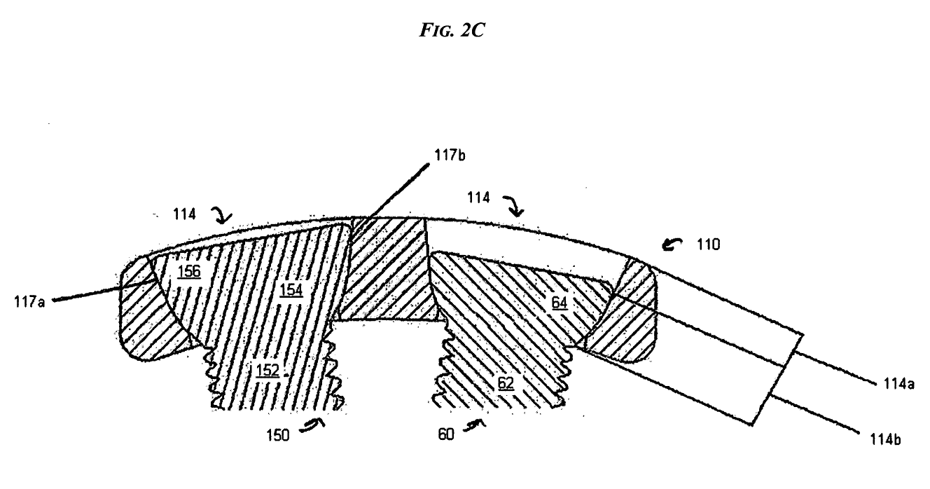

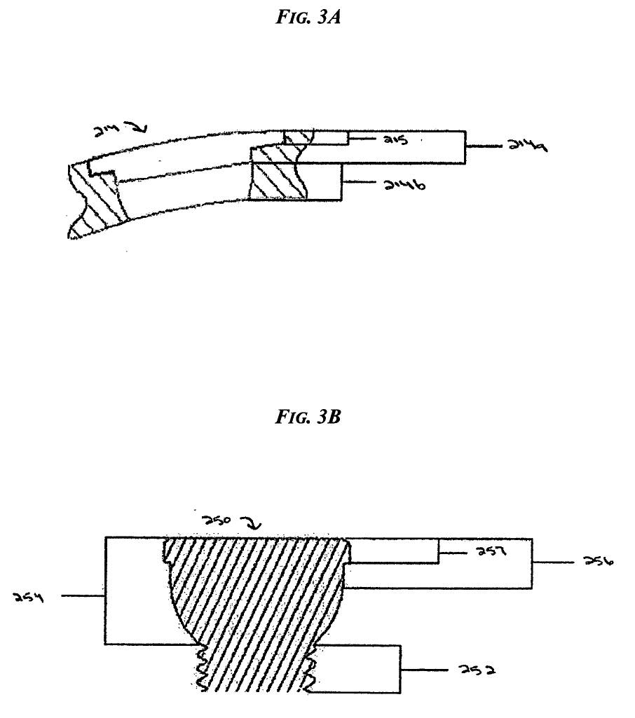

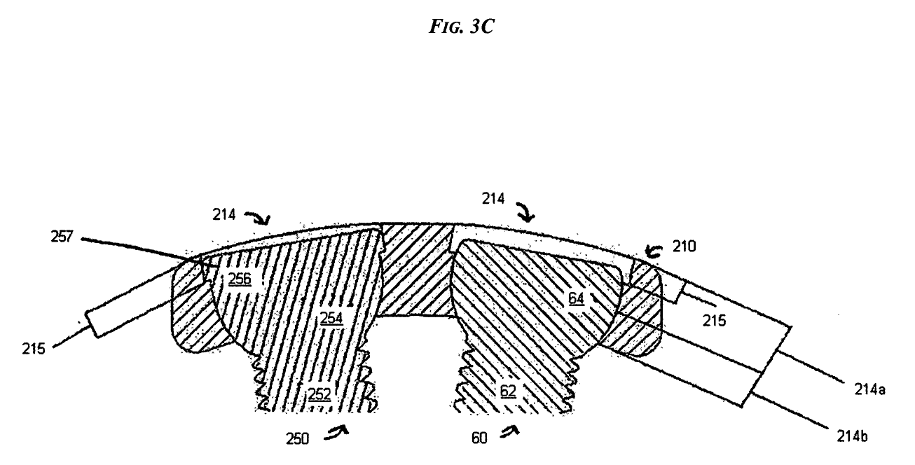

As previously indicated, an exemplary spinal plating system can have a variety of other configurations to allow a thru-bore to interchangeably receive a variable angle bone screw and a fixed angle bone screw. By way of non-limiting example, FIGS. 2A-2C and FIGS. 3A-3C illustrate additional exemplary embodiments. In the embodiment shown in FIGS. 2A-2B, the thru-bore 114 can be similar to thru-bore 14 shown in FIG. 1B, and the head 154 of the fixed angle bone screw 150 can be similar to the head 54 of the fixed angle bone screw 50, however the thru-bore 114 and the head 154 can each have a proximal region 114a, 156 that differ in shape from the embodiment shown in FIGS. 1B and 1C. In this embodiment, the proximal region 114a of the thru-bore 114 is substantially conical such that the opposed walls 117a, 117b of the proximal region 114a have a diameter D_{c} which decreases in a proximal to distal direction. The exemplary fixed angle bone screw 150, as shown in FIG. 2B, can likewise have a screw head 154 with a proximal region 156 that is substantially conical. As a result, when the head 154 is disposed within the thru-bore 114, as shown in FIG. 2C, the substantially conical proximal region 156 of the head 154 will engage the substantially conical proximal region 114a of the thru-bore 114, thereby preventing movement of the bone screw 150, and in particular the shank 152, relative to the plate 110. As with the embodiment shown in FIGS. 1A-1E, the thru-bore 114 can also receive the variable angle bone screw 60 shown in FIG. 1D. As noted above, the head 64 of the variable angle bone screw 60 can rest only within the distal region 114b of the thru-bore 114, as shown in FIG. 2C, or it can have a proximal region (not shown) with a diameter that is smaller than the diameter of the proximal region 114a of the thru-bore 114 to allow the screw head 64 to pivot within the thru-bore 114, thereby allowing polyaxial movement of the shank 62 relative to the plate 110.

In another exemplary embodiment, shown in FIG. 3A, the proximal region 214a of the thru-bore 214 can include a cut-out portion, such as a flange-receiving recess 215, formed therein for receiving a corresponding protrusion, such as flange 257, of the fixed angle screw head 254. While the flange-receiving recess 215 can have any shape, such as rectangular, square, oval, etc., in the illustrated embodiment the flange-receiving recess 215 is cylindrical. The flange-receiving recess 215 can also have any size and it can occupy a portion or all of the proximal region 214a of the thru-bore 214. The exemplary fixed angle bone screw 250, as shown in FIG. 3B, can likewise have a flange or lip 257 formed on the proximal-most portion of the proximal region 256 of the head 254. The flange 257 can have any shape and size that corresponds to the shape and size of the flange-receiving recess 215, however, as shown in the exemplary embodiment, the flange 257 is cylindrical. As a result, when the head 254 is disposed within the thru-bore 214, as shown in FIG. 3C, the flange 257 on the proximal region 256 of the head 254 will sit within the flange-receiving recess 215 in the proximal region 114a of the thru-bore 114, thereby preventing movement of the bone screw 250, and in particular the shank 252, relative to the plate 210. As with the embodiment shown in FIGS. 1A-1E, the thru-bore 214 can also receive the variable angle bone screw 60 shown in FIG. 1D. As noted above, the head 64 of the variable angle bone screw 60 can rest only within the distal region 214b of the thru-bore 214, as shown in FIG. 3C, or it can have a proximal region (not shown) with a diameter that is smaller than the diameter of the proximal region 214a of the thru-bore 214, or that otherwise does not extend into the flange-receiving recess 215 such that the screw head 64 can pivot within the thru-bore 214 to allow polyaxial movement of the shank 62 relative to the plate 210.

A person skilled in the art will appreciate that the proximal region of a thru-bore in an exemplary spinal plate can have a variety of other configurations and shapes to allow angular movement of a variable angle bone screw, and to substantially prevent movement of a fixed angle bone screw. By way of non-limiting example, the proximal region of the thru-bore can include cut-out portions or surface features that are adapted to receive or engage corresponding surface features or cut out portions of a fixed angle bone screw, and that do not interfere with movement of a variable angle bone screw.

While not illustrated, the various embodiments of the spinal plating systems disclosed herein can also include a locking or retaining mechanism for preventing bone screw backout. In one exemplary embodiment, the locking mechanism can be integrated into the screw head, as described in a U.S. Patent filed on even date herewith and entitled “Locking Bone Screw and Spinal Plate System” of Gorhan et al., which is incorporated by reference herein in its entirety. In another exemplary embodiment, as shown in FIG. 1A, the locking mechanism can be integrated onto the surface of the plate 10. The integrated locking mechanism can be, for example, a cam 13 that is rotatable between an unlocked position and a locked position, in which the cam 13 is forced against the head of the bone screw to provide bone screw backout resistance. An exemplary cam-type locking mechanism is described in U.S. Pat. No. 5,549,612 of Yapp et al. entitled “Osteosynthesis Plate System,” which is also incorporated by reference herein in its entirety. Other exemplary retaining or locking mechanisms include, by way of non-limiting example, locking washers, locking screws, and bone screw covers. One skilled in the art will appreciate that various combinations of locking mechanisms can be used as well. Other exemplary locking mechanisms are disclosed in U.S. Pat. No. 6,331,179 to Fried et al., U.S. Pat. No. 6,159,213 to Rogozinski; U.S. Pat. No. 6,017,345 to Richelsoph; U.S. Pat. No. 5,676,666 to Oxiand et al.; U.S. Pat. No. 5,616,144 to Yapp et al.; U.S. Pat. No. 5,261,910 to Warden et al.; and U.S. Pat. No. 4,696,290 to Steffee.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A spinal plate system, comprising:
a spinal plate having a thru-bore formed therein with a proximal region and a distal region;
a variable angle bone screw having a shank sized to allow passage thereof through the thru-bore in the spinal plate, and a head formed on a proximal end of the shank and receivable within the thru-bore in the spinal plate, the head being adapted to allow angular movement of the shank when the head is fully seated within the thru-bore in the spinal plate; and
a fixed angle bone screw having a shank sized to allow passage thereof through the thru-bore in the spinal plate, and a head formed on a proximal end of the shank, the head including a proximal region and a distal region receivable within the proximal region and the distal region of the thru-bore in the spinal plate, the proximal region of the head of the fixed angle bone screw being adapted to engage the proximal region of the thru-bore in the plate to fix a position of the shank relative to the spinal fixation plate when the head is fully seated within the thru-bore in the spinal plate.

2. The spinal plate system of claim 1, wherein the distal region of the thru-bore is substantially spherical.

3. The spinal plate system of claim 2, wherein the head of the variable angle bone screw is substantially spherical and is adapted to be received within at least a portion of the thru-bore in the spinal plate.

4. The spinal plate system of claim 2, wherein the distal region of the head of the fixed angle bone screw is substantially spherical, and the proximal region of the head of the fixed angle bone screw has a shape that complements a shape of the proximal region of the thru-bore.

5. The spinal plate system of claim 2, wherein the proximal region of the thru-bore in the plate is substantially cylindrical, and the proximal region of the head of the fixed angle bone screw is substantially cylindrical.

6. The spinal plate system of claim 2, wherein the proximal region of the thru-bore in the plate is substantially conical, and the proximal region of the head of the fixed angle bone screw is substantially conical.

7. The spinal plate system of claim 2, wherein the proximal region of the thru-bore includes a flange-receiving recess formed therein, and the proximal region of the head of the fixed angle bone screw includes a flange adapted to be received in the flange-receiving recess.

8. The spinal fixation system of claim 1, wherein the head of the variable angle bone screw is substantially spherical and the head of the fixed angle bone screw has a distal region that is substantially spherical and a proximal region that is non-spherical.

9. A spinal plate system, comprising:
a spinal fixation plate having a thru-bore formed therein and extending between a bone-contacting surface and a non-bone-contacting surface thereof;
a first bone screw having a head that is receivable within the thru-bore in the spinal fixation plate, and a shank that is angularly variable when the head is seated within the thru-bore formed in the spinal plate; and
a second bone screw having a head that is receivable within the thru-bore in the spinal fixation plate, and a shank, the head including a proximal region and a distal region that differ in shape relative to one another, the proximal region having a shape that is complementary to a shape of a proximal region of the thru-bore in the spinal fixation plate to engage the thru-bore such that the shank is angularly fixed relative to the spinal fixation plate.

10. The spinal plate system of claim 9, wherein a proximal region of the thru-bore is non-spherical and a distal region of the thru-bore is substantially spherical.

11. The spinal plate system of claim 10, wherein the head of the first bone screw is substantially spherical and is adapted to be received within at least a portion of the thru-bore in the spinal plate.

12. The spinal plate system of claim 10, wherein the distal region of the head of the second bone screw is substantially spherical, and the proximal region of the head of the second bone screw has a shape that complements a shape of the proximal region of the thru-bore.

13. The spinal plate system of claim 10, wherein the proximal region of the thru-bore in the plate is substantially cylindrical, and the proximal region of the head of the second bone screw is substantially cylindrical.

14. The spinal plate system of claim 10, wherein the proximal region of the thru-bore in the plate is substantially conical, and the proximal region of the head of the second bone screw is substantially conical.

15. The spinal plate system of claim 10, wherein the proximal region of the thru-bore includes a flange-receiving recess formed therein, and the proximal region of the head of the second bone screw includes a flange adapted to be received in the flange-receiving recess.

16. A spinal fixation system of claim 9, wherein the head of the first bone screw is substantially spherical and the head of the second bone screw head has a distal region that is substantially spherical and a proximal region that is non-spherical.

17. A spinal fixation kit, comprising:
a fixed angle bone screw having a head and a shank, the head including a distal region having a substantially spherical shape, and a proximal region having a non-spherical shape that is adapted to engage a proximal region of a thru-bore formed in a spinal fixation plate to maintain the shank in a fixed position relative to the plate; and
a variable angle bone screw having a head and a shank, the head being adapted to sit within a thru-bore in a spinal fixation plate and to allow angular movement of the shank with respect to the spinal fixation plate;
wherein the fixed angle bone screw and the variable angle bone screw are adapted to interchangeably fit within the same thru-bore in a spinal fixation plate.

18. The spinal fixation kit of claim 17, wherein the head of the variable angle bone screw is substantially spherical.

19. The spinal fixation kit of claim 17, wherein the proximal region of the head of the fixed angle bone screw is substantially cylindrical.

20. The spinal fixation kit of claim 17, wherein the proximal region of the head of the fixed angle bone screw is substantially conical.

21. The spinal fixation kit of claim 17, wherein the proximal region of the head of the fixed angle bone screw includes a flange.

22. The spinal fixation kit of claim 17, further comprising a plate having a thru-bore formed therein.

23. The spinal fixation kit of claim 22, wherein the thru-bore includes proximal and distal regions, and wherein the proximal region of the head of the fixed angle bone screw has a shape that complements a shape of the proximal region of the thru-bore.

24. The spinal fixation kit of claim 23, wherein the proximal region of the thru-bore and the proximal region of the head of the fixed angle bone screw are substantially cylindrical.

25. The spinal fixation kit of claim 23, wherein the proximal region of the thru-bore and the proximal region of the head of the fixed angle bone screw are substantially conical.

26. The spinal fixation kit of claim 23, wherein the proximal region of the thru-bore includes a flange-receiving recess and the proximal region of the fixed angle bone screw includes a flange adapted to be received in the flange-receiving recess.

27. A spinal plate system, comprising:
a spinal plate having a thru-bore formed therein;
a first bone screw insertable through the thru-bore to engage the spinal plate in any one of a plurality of angles relative to the spinal plate; and
a second bone screw insertable through the thru-bore to engage the spinal plate at a fixed angle relative to the spinal plate, the second bone screw having a head including a proximal region configured to engage at least a portion of the thru-bore to fix the second bone screw at the fixed angle.

28. The spinal plate system of claim 27, wherein the proximal region of the head of the second bone screw has a size and shape approximate to a size and shape of a proximal region of the thru-bore.
